(19) United States
(12) Patent Application Publication
(10) Pub. No.: US 2015/0230903 A1
(43) Pub. Date: Aug. 20, 2015

(54) DEVICE FOR DETECTING AND INDICATING THE PREPARATORY PHASE OF BIRTHING A BOVINE OR EQUINE ANIMAL, AND CORRESPONDING METHOD AND COMPUTER PROGRAM PRODUCT

(71) Applicant: Evolution NT, Blain (FR)

(72) Inventor: Jean-Michel Philipot, Betton (FR)

(21) Appl. No.: 14/425,272

(22) PCT Filed: Aug. 28, 2013
(86) PCT No.: PCT/FR2013/051982

§ 371 Date: Mar. 2, 2015

(30) Foreign Application Priority Data
Aug. 31, 2012 (FR) 12 58128
Sep. 11, 2012 (FR) 12 58539

(51) Int. Cl. A61D 17/00; A61B 5/11; A61B 5/00

(52) U.S. Cl. CPC A61D 17/008; A61B 5/4356; A61B 5/1114; A61B 5/1118; A61B 5/4255; A61B 5/7246; A61B 5/7282; A61B 5/746; A61B 5/0011; A61B 5/002; A61B 5/1122; A61B 2503/40; A61B 2560/0214; A61B 2562/164; A61B 2560/0475; A61B 2562/0219

(57) ABSTRACT

A device and a method are provided for detecting and indicating a preparatory phase of birthing a bovine or equine animal. The device is configured to be attached to the tail of the bovine or equine animal. The device contains elements configured to: measure components of acceleration of the device according to at least one axis, such as three orthogonal axes; process the measurements of the components, to detect an occurrence of the preparatory phase; generate a radio communication signal configured in case the occurrence is detected; and transmit the generated signal to a collector forming a relay or to a terminal.

### 1. CROSS-REFERENCE TO RELATED APPLICATIONS

This Application is a Section 371 National Stage Application of International Application No. PCT/FR2013/051982, filed Aug. 28, 2013, the content of which is incorporated herein by reference in its entirety, and published as WO 2014/033403 on Mar. 6, 2014, not in English.

### 2. FIELD OF THE INVENTION

The field of the invention is that of agriculture, and specifically the monitoring of bovine or equine animals.

More specifically, the invention relates to a device and a corresponding method for detecting and indicating the preparatory phase of birthing a bovine or equine animal.

### 3. PRIOR ART

It is well known that, without human intervention, some cows or mares lose their newborns at birth.

It is therefore necessary for a breeder to monitor full term animals, both day and night, to be able to intervene if needed during parturition to attempt to save the newborn or reduce postpartum complications.

In addition, it is common practice to perform several services simultaneously within the same livestock, which complicates the breeder's monitoring task insofar as calvings or foalings are produced over the same period.

In order to prevent a breeder from having to remain continuously mobilized both night and day, and to allow him to rest during the calving or foaling period, various techniques are proposed.

Calving detection techniques are known, consisting of introducing sensors measuring the temperature, pressure and/or acidity inside the reproductive system of a cow in order to identify the immediacy of birthing. For example, one of these techniques consists of introducing a thermometer into the vaginal canal of the animal, which will be expelled when the waters break.

One disadvantage of these intrusive techniques is that the introduction and positioning of sensors into the reproductive system of the cow is delicate, and is a source of infection and/or irritation for the cow.

Another disadvantage of these intrusive techniques is that normal metabolic changes in the animal due to its activity may trigger false alerts.

Still another disadvantage of these intrusive techniques is that they are expensive to implement.

Attaching a mercury inclinometer or ball inclinometer on the tail of a cow or mare has also been proposed, which will trigger an alarm when birthing approaches, when the tail of the animal remains raised.

One disadvantage of this technique is that it poses reliability problems. In fact, it has been observed that alerts can be triggered at the wrong time, for example when the animal changes position or lies down.

Girthing the abdomen of a cow with a belt that measures the uterine activity of the animal and transmits an alarm when the frequency of contractions increases has also been proposed.

This technique presents several disadvantages: The belt presents a danger to the animal if it is too tight. However, the belt should not be too loose or it risks not functioning correctly, which would require it to be retightened every 3 to 4 hours. In addition, this technique is expensive and in certain cases does not enable deformations due to the animal's labor to be distinguished from other deformations resulting from the animal's normal activities when eating or moving about.

### 4. DISCLOSURE OF THE INVENTION

These objectives, as well as others that will appear subsequently, are reached by using a device for detecting and indicating the preparatory phase of birthing a bovine or equine animal, that is capable of being attached to the tail of said bovine or said equine animal.

Firstly, it should be noted that, in general, placing a device on the tail of an animal is an easy operation, which constitutes an advantage of the detecting and indicating device according to the invention.

In the context of the invention, the expression “preparatory phase of birthing” is understood in its general meaning. Therefore, it is the phase preceding the engagement of the fetus in the pelvis, with a duration of between 2 and 24 hours, during which the first uterine contractions and dilation of the cervix occur.

According to the invention, such a detecting and indicating device comprises:
means for measuring the components of acceleration of said device according to at least one axis, and preferably according to three orthogonal axes;
means for processing the measurements of said components, intended to detect an occurrence of said preparatory phase;
means for generating a radio communication signal configured so as to generate said signal in case said occurrence is detected;
means for transmitting said generated signal to a collector forming a relay or to a terminal.

Therefore, in an original way, the invention proposes exploiting the acceleration measurements of the tail of a cow, mare or jennet to detect the start of parturition.

The device according to the invention cleverly presents means for processing integrated measurements, which enables its energy consumption to be limited due to the fact that the quantity of signals that it transmits externally is very small, thereby conferring it a particularly high energy self-sufficiency.

According to one particular aspect of the invention, said radio communication signal is a digital signal.

Therefore, subsequently converting this signal is unnecessary.

In one particularly advantageous embodiment of the invention, an identifier of said device is encapsulated in said digital signal.

In this way, the breeder can be alerted to the identity of the animal whose birthing is imminent.

Preferably, said processing means comprise means for comparing a sequence of said measurements with at least one sequence of reference data representative of a physiological or behavioral manifestation of said bovine or equine animal belonging to the group comprising:
muscle contractions of the uterus;
colic resulting from uterine pain;
raising of the tail;
hyperactivity.

Therefore, by correlating the measurements of acceleration components as a function of one or more physiological or behavioral manifestations of the preparatory birthing phase, it is possible to detect the beginning of this phase precisely and to rule out measurements that have no connection with the physiological or behavioral manifestations resulting from birthing.

In fact, the inventors have observed that, in animals, uterine contractions, colic, the tail being raised and staying raised for several seconds and hyperactivity constitute the most characteristic manifestations that may be observed through the animal's tail movements during the preparatory birthing phase, and that each of these manifestations would trigger a specific and reproducible sequence of movements at least over one portion of a bovine or equine animal population. The inventors have also observed that these specific sequences may vary between two breeds of cows or mares.

Even more preferably, said processing means comprise means for comparing a sequence of said measurements with two, three or four reference sequences of data, each representative of a different physiological or behavioral manifestation of said bovine or equine animal belonging to the group comprising:
muscle contractions of the uterus;
colic resulting from uterine pain;
raising of the tail;
hyperactivity.

Therefore for example, during processing it may be provided to compare the measurement sequence to a reference sequence representative of uterine contractions and to a reference sequence representative of colic resulting from uterine pain or to a reference sequence representative of uterine contractions, to a reference sequence representative of tail raising and to a reference sequence representative of hyperactivity.

According to a preferential aspect of the invention, said reference sequence of data presents, at least for one of its components, at least 3 spikes, preferentially from 5 to 10 spikes, spaced over a duration of 3 to 4 seconds.

In fact, the inventors observed that such a data sequence was substantially universal in all animals.

Advantageously, said processing means comprise means for determining the correlation coefficient or coefficients between said measurement sequence and said reference sequence or sequences.

Thus the differences between the measuring sequence and the reference sequence or sequences can be simply quantified.

In at least one embodiment of the invention, said processing means comprise means for comparing said correlation coefficient or a product of said correlation coefficients given a weighting with a predetermined threshold value, and means for generating alert data indicating that an occurrence of said preparatory phase has been detected when the result of said comparison indicates that said correlation coefficient or said product of said correlation coefficients given a weighting is equal to or greater than said predetermined threshold value.

According to a particular embodiment of the invention, the weighting of each of said correlation coefficients in said product is equal to 1.

The sensitivity of the detection of each of the physiological or behavioral manifestations is thus identical.

Preferably, a detecting and indicating device such as those described above comprises an autonomous device for supplying said measuring means and/or said processing means and/or said signal generating means and/or said transmission means with electrical power.

Therefore an energy self-sufficient device is obtained.

Preferentially, a detecting and indicating device such as those described above comprises a housing presenting a lower portion configured to conform to the shape of said tail.

Therefore, a suitable attachment of the device onto the tail is obtained.

This lower portion of the housing may, in particular, present a substantially conical shape.

Advantageously, said signal transmitted by said transmission means has a range of between 150 and 500 meters.

Therefore, the signal may be transmitted to a relay situated in, on near, the cow barn or stable where the animal is penned, regardless of where the animal is located in the building. The signal may then be transmitted directly over a communication terminal for the breeder, such as a portable telephone or a personal assistant for example, if the terminal is found near the building.

The invention also relates to a method for detecting the preparatory phase of birthing a bovine or equine animal.

According to the invention, such a method comprises the following steps:
measuring data representative of the acceleration components of a portion of the tail of said bovine or equine animal according to at least one axis, and preferentially according to three orthogonal axes;
processing the measurements of said components intended to detect an occurrence of said preparatory phase, comprising a step of comparing a sequence of said measurements with at least one sequence of reference data representative of a physiological or behavioral manifestation of said bovine or equine animal belonging to the group comprising:
muscle contractions of the uterus;
colic resulting from uterine pain;
raising of the tail;
hyperactivity.

Advantageously, said processing step comprises:
a step of determining the correlation coefficient or coefficients between said measurement sequence and said reference sequence or sequences;
a step of comparing said correlation coefficient or a product of said correlation coefficients given a weighting with a predetermined threshold value, so as to be able to generate an alert signal intended to alert that an occurrence of said preparatory phase has been detected, when the result of said comparison indicates that said correlation coefficient or said product of said correlation coefficients given a weighting is equal to or greater than said predetermined threshold value.

The invention also relates to a computer program product downloadable from a communication network and/or recorded on a computer readable medium and/or executable by a processor, comprising program code instructions for implementing the step of processing the detecting method described above, when said program is executed by a computer.

### 5. LIST OF FIGURES

Other characteristics and advantages of the invention will appear more clearly upon reading the following description of an embodiment of the invention, given by way of a simple, non-limiting illustrative example, and of the attached drawings, among which:

FIG. 1 is a schematic view of a device according to the invention attached to the tail of a cow;

FIG. 2 is a detail view of the device presented in reference to FIG. 1;

FIG. 3 is an illustration of variations of the acceleration component according to the vertical direction during a typical uterine contraction sequence;

FIG. 4 is an illustration in graphic form, and over time, of an example of the result from processing the measurements carried out within the device presented in reference to FIG. 1.

### 6. DETAILED DESCRIPTION OF AN EXAMPLE OF EMBODIMENT OF THE INVENTION

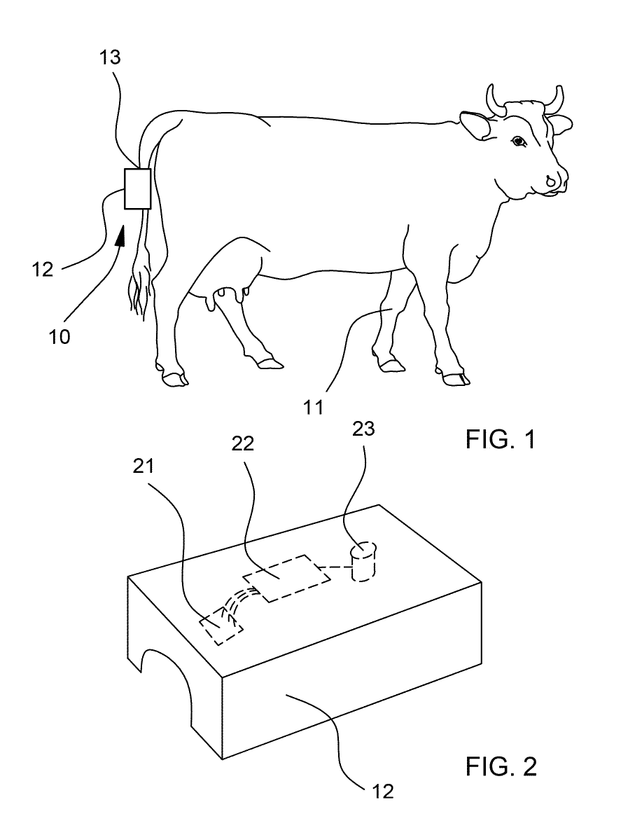

A detecting and indicating device 10 according to the invention is represented in FIG. 1, attached to the tail of a cow 11.

The device is present in the form of a sealed housing 12 which lower face 13 is in a substantially conical shape so as to conform to the shape of the tail of cow 11.

To attach housing 12, an adhesive substance is advantageously applied, preferentially an adhesive used to attach KAMAR (registered trademark) type labels, onto the tail of the animal, then the lower face of the housing is pressed against this adhesive surface and lastly a layer of adhesive tape is wound around both the housing and the tail of the animal.

As may be seen in FIG. 2, which is a detail view of device 10, the housing 12 includes a three-dimensional accelerometer 21 of the electromechanical microsystem type connected to an electronic card 22 equipped with an MSP 430 microcontroller, which processes the measurements provided by accelerometer 21, and an integrated circuit ensuring optimization of the power consumed by the housing.

Inside housing 12, there is an antenna 23, enabling a digital signal conforming to the GSM standard to be transmitted to a relay or to a portable telephone and an L5 battery, which powers the accelerometer, card 22 and antenna 23.

The microcontroller equipping card 22 processes the measurement sequences issued by accelerometer 21 and generates an alert signal when the preparatory birthing phase is detected, which signal is transmitted by antenna 23.

In this mode of embodiment of the invention, sequences constituted of 3 groups of 64 measurement points sampled at 16 Hz, each corresponding to variations in an acceleration component of housing 12 for a period of 4 seconds, are continuously processed by the microcontroller.

These measurement sequences are stored as produced in a random access memory of the card 22 and are not kept after processing. In fact, as the processing device is provided to be able to be operated continuously over several years, and more specifically for 5 to 10 years in this particular embodiment of the invention, providing a memory with sufficient storage capacity is not possible.

Correlation coefficients of each of these sequences of measurements of components of housing's acceleration according to 3 axes, with four predetermined reference sequences of data respectively representative of uterus muscle contractions, colic resulting from uterine pain, tail raising or hyperactivity are successively evaluated by the microcontroller.

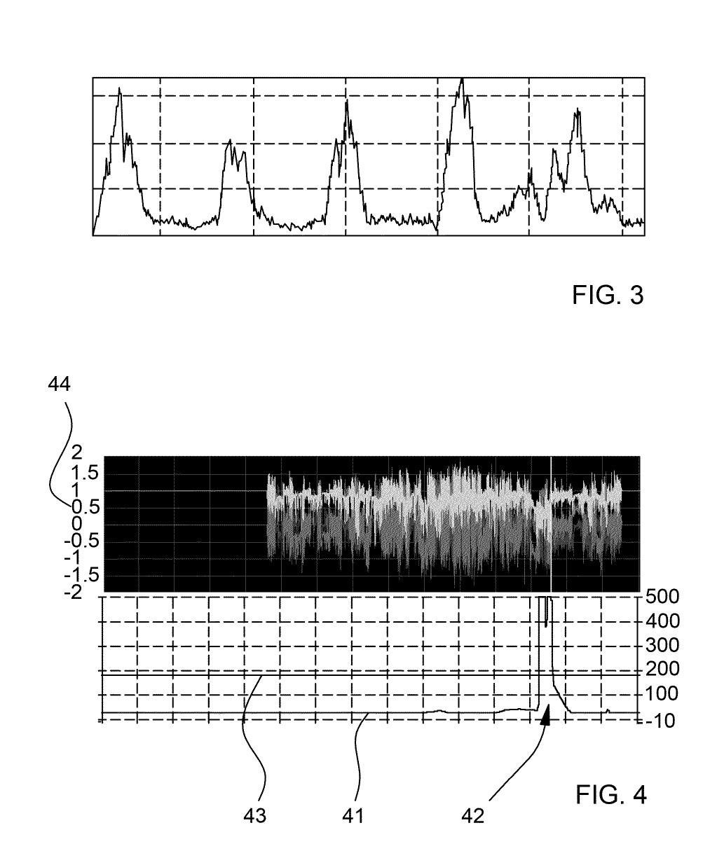

By way of illustration, FIG. 3 represents a reference sequence corresponding to variations in the acceleration component according to the vertical direction when muscle contractions of the uterus occur.

In the event where one of the correlation coefficients turns out to be less than a predetermined minimum value specific to this coefficient, said minimum value is assigned to it.

The microcontroller then assigns a predetermined weighting to each of the correlation coefficients, and then multiplies the weighted coefficients to each other, which provides an aggregate index representative of the animal's condition.

Therefore, for example, in this mode of embodiment of the invention, a weighting of 3 and 1.6 is respectively given to the correlation coefficients relative to uterine contractions and to tail raising and a value of 1 is given to correlation coefficients relative to colic resulting from uterine pain and to hyperactivity, in order to increase detection sensitivity and to be able to guarantee a detection rate of the beginning of the preparatory phase equal to or greater than 80%.

In variations of this mode of embodiment of the invention, only considering one, two or three correlation coefficients to determine the aggregate index representative of the animal's condition and therefore only taking one, two or three physiological or behavioral manifestations into account to detect the beginning of the preparatory birthing phase may also be provided.

The aggregate index representative of the animal's condition is finally compared to a predetermined threshold value. In the event where the index value exceeds this predetermined threshold value, an alert signal intended to alert the breeder that an occurrence of preparatory phase of birthing has been detected is generated by the microcontroller and transmitted by antenna 23.

Variations in the aggregate index 41 according to time over a period preceding a preparatory birthing phase up to detection, indicated by significant spike 42, are illustrated in FIG. 4. The predetermined threshold is represented by horizontal line 43 in this FIG. 4.

In the upper frame 44 of FIG. 4 are represented variations according to time of components along 3 orthogonal axes X, Y and Z of the acceleration of device 10 measured by accelerometer 21.

An exemplary embodiment of the present disclosure provides a technique for detecting and indicating the preparatory phase of birthing a bovine or equine animal that is reliable.

At least one particular embodiment proposes such a detecting and indicating technique that presents a rate of detection of the preparatory phase of birthing greater than 80%.

At least one particular embodiment provides such a detecting and indicating technique that is simple to implement and has a reduced cost.

At least one particular embodiment proposes such a detecting and indicating technique that is reusable.

At least one particular embodiment provides a technique for detecting and indicating the preparatory phase of birthing a bovine or an equine animal that requires little maintenance and that is energy self-sufficient over a period of several years.

Although the present disclosure has been described with reference to one or more examples, workers skilled in the art will recognize that changes may be made in form and detail without departing from the scope of the disclosure and/or the appended claims.

1. A device for detecting and indicating a preparatory phase of birthing a bovine or equine animal, capable of being attached to the tail of said bovine or equine animal,
wherein the device comprises:
means for measuring the components of acceleration of said device according to at least one axis;
means for processing the measurements of said components, to detect an occurrence of said preparatory phase;
means for generating a radio communication signal configured so as to generate said signal in case said occurrence is detected;
means for transmitting said generated signal to a collector forming a relay or to a terminal.

2. The device according to claim 1, wherein said radio communication signal is a digital signal.

3. The device according to claim 2, wherein an identifier of said device is encapsulated in said digital signal.

4. The device according to claim 1, wherein said processing means comprise means for comparing a sequence of said measurements with at least one sequence of reference data representative of a physiological or behavioral manifestation of said bovine or equine animal belonging to the group consisting of:
muscle contractions of the uterus;
colic resulting from uterine pain;
raising of the tail;
hyperactivity.

5. The device according to claim 4, wherein said reference sequence of data presents, at least for one of its components, at least 3 spikes spaced over a duration of 3 to 4 seconds.

6. The device according to claim 4, wherein said processing means comprise means for determining the correlation coefficient or coefficients between said sequence of measurements and said reference sequence or sequences.

7. The device according to claim 6, wherein said processing means comprise means for comparing said correlation coefficient or a product of said correlation coefficients given a weighting with a predetermined threshold value, and means for generating alert data indicating that an occurrence of said preparatory phase has been detected when the result of said comparison indicates that said correlation coefficient or said product of said correlation coefficients given a weighting is equal to or greater than said predetermined threshold value.

8. The device according to claim 7, wherein the weighting of each of said correlation coefficients in said product is equal to 1.

9. The device according to claim 1, wherein the device comprises an autonomous device for supplying said measuring means and/or said processing means and/or said signal generating means and/or said transmission means with electrical power.

10. The device according to claim 1, wherein the device comprises a housing presenting a lower portion configured to conform to the shape of said tail.

11. The device according to claim 1, wherein said signal transmitted by said transmission means has a range of between 150 and 500 meters.

12. A method of detecting a preparatory phase of birthing a bovine or equine animal, wherein said method comprises
measuring data representative of the acceleration components of a portion of the tail of said bovine or equine animal according to at least one axis;
processing the measurements of said components intended to detect an occurrence of said preparatory phase, comprising comparing a sequence of said measurements with at least one sequence of reference data representative of a physiological or behavioral manifestation of said bovine or equine animal belonging to the group comprising:
muscle contractions of the uterus;
colic resulting from uterine pain;
raising of the tail;
hyperactivity.

13. The method according to claim 12, wherein said processing comprises:
determining a correlation coefficient or coefficients between said measurement sequence and said reference sequence or sequences;
comparing said correlation coefficient or a product of said correlation coefficients given a weighting with a predetermined threshold value, so as to be able to generate an alert signal to alert that an occurrence of said preparatory phase has been detected, when the result of said comparison indicates that said correlation coefficient or said product of said correlation coefficients given a weighting is equal to or greater than said predetermined threshold value.

14. A non-transitory computer-readable medium comprising a computer program product stored thereon and executable by a processor, wherein the product comprises program code instructions for implementing a method of detecting a preparatory phase of birthing a bovine or equine animal, when said program is executed by the processor, wherein the method comprises:
receiving and storing measured data representative of the acceleration components of a portion of the tail of said bovine or equine animal according to at least one axis;
processing the measured data of said components to detect an occurrence of said preparatory phase, comprising comparing a sequence of said measurements with at least one sequence of reference data representative of a physiological or behavioral manifestation of said bovine or equine animal belonging to the group comprising:
muscle contractions of the uterus;
colic resulting from uterine pain;
raising of the tail;
hyperactivity.

15. The device of claim 1, wherein the means for measuring are configured to measure the components of acceleration of said device according to three orthogonal axes.

16. The device of claim 4, wherein the means for comparing compare the sequence of said measurements with two, three or four reference sequences of data, each representative of a different physiological or behavioral manifestation of said bovine or equine animal belonging to the group consisting of:
muscle contractions of the uterus;
colic resulting from uterine pain;
raising of the tail;
hyperactivity.

17. The device according to claim 5, wherein said reference sequence of data presents, at least for one of its components, from 5 to 10 spikes spaced over the duration of 3 to 4 seconds.

18. The method of claim 12, wherein measuring comprises measuring the data representative of the acceleration components according to three orthogonal axes.
